(19) United States
(12) Patent Application Publication
(10) Pub. No.: US 2010/0098779 A1
(43) Pub. Date: Apr. 22, 2010

(54) COMPOSITIONS AND METHODS FOR PROPHYLACTIC AND THERAPEUTIC SUPPLEMENTATION OF NUTRITION IN SUBJECTS

(75) Inventors: Charles J. Balzer, West Orange, NJ (US); John A. Giordano, West Orange, NJ (US)

Correspondence Address: DON J. PELTO;Sheppard, Mullin, Richter & Hampton LLP, 1300 I STREET, NW, 11TH FLOOR EAST, WASHINGTON, DC, 20005, US

(73) Assignee: Everett Laboratories, Inc.

(21) Appl. No.: 12/256,007

(22) Filed: Oct. 22, 2008

(51) Int. Cl. A61K 33/30; A23L 1/303

(52) U.S. Cl. 424/641; 426/73

(57) ABSTRACT

The present invention relates to compositions and methods for prophylactic nutritional supplementation and therapeutic nutritional supplementation. Specifically, the composition provides a nutritional supplement with a beneficial increased supplementation of vitamin D and the method involves administering to an individual a composition comprising carotenoids, vitamin E, vitamin D, vitamin C, thiamine, riboflavin, niacin, folic acid, pyridoxine, biotin, pantothenic acid, cobalamin, magnesium, manganese, zinc, selenium, chromium, copper, alpha lipoic acid, and lutein.

### FIELD OF THE INVENTION

The present invention relates to compositions comprising various vitamins, minerals, antioxidants and nutrients, and methods for using these compositions for prophylactic nutritional supplementation and therapeutic nutritional supplementation. Specifically, the present invention relates to providing nutritional and therapeutic nutritional supplementation of physiologically stressful conditions and, for example, providing a beneficial increased supplementation of vitamin D for conditions related to vitamin D deficiency.

### BACKGROUND OF THE INVENTION

Nutrition plays a critical role in maintaining good health. Proper nutrition prevents dietary deficiencies, and also protects against the development of disease. Proper nutrition plays an increasingly important role as the body faces physiological stress. For example, as the body ages it suffers significant physiological stresses. Specifically, as the body metabolism shifts to accumulating larger fat stores and decreasing lean body mass, this increase in body weight may lead to obesity and associated conditions such as diabetes, cardiovascular disease, hypertension, osteoarthritis, and cancer. Other conditions, such as anorexia, malnutrition, gastrointestinal disorders, chronic alcoholism, chronic infections, acute infections, congestive heart failure, hyperthyroidism, poorly controlled diabetes, cheilosis, gingivitis, stomatitis and dietary restrictions, often result in physiological stresses that may be exacerbated by poor nutrition. In particular, these disease states may result in increased oxidative stress or elevated homocysteine levels that further compromise health. Thus, nutritional supplementation serves a vital role in protecting against poor nutrition and disease.

Nutritional supplementation, however, is an evolving science. As research is determining the beneficial effects of nutritional supplements are findings that the recommended daily allowance of some nutrients and vitamins may still not be sufficient. For example, recent research suggests that vitamin D has more positive physiological effects than previous thought. It has recently been determined that vitamin D has previously unknown roles in the enhancement of vascular function, defense against cancer, immuno-competence, blood pressure regulation and possessing the ability to enhance cellular insulin sensitivity in the human body.

Due to the additional roles that vitamin D plays in the human body, it has recently been determined that higher daily vitamin D intake beyond current recommendations may be associated with better health outcomes. Indeed, studies suggest increasing the serum level of 25-hydroxyvitamin D, a beneficial derivative of vitamin D, to a 30 ng/ml serum range. A 30 ng/ml appears to be the most advantageous serum level in recent studies reviewing patient bone mineral density (BMD), lower extremity function, dental health, risk of falls, admission to nursing home, fractures, cancer prevention and incident hypertension.

Current nutritional supplements, however, do not include high enough dosage amounts to obtain such a high serum level of 25-hydroxyvitamin D in patients. A nutritional supplement that includes a higher dosage amount of vitamin D is therefore currently needed.

Thus, the invention herein provides for compositions and methods, designed to optimize health and wellness, minimize oxidative stress, and provide a beneficial increased supplementation of vitamin D.

### SUMMARY OF THE INVENTION

The present invention provides nutritional compositions and methods of using these compositions for both prophylactic and therapeutic nutritional supplementation, specifically in physiologically stressful conditions. The present invention also relates to novel compositions of vitamins and minerals that can be used to supplement nutritional deficiencies of vitamin D observed in patients and illnesses and health issues such as anorexia, malnutrition, gastrointestinal disorders, chronic alcoholism, chronic infections, acute infections, congestive heart failure, hyperthyroidism, poorly controlled diabetes, cheilosis, gingivitis, stomatitis, and/or dietary restrictions. In addition, the compositions may be used to treat the nutritional deficiencies of patients suffering from a disease state that results in increased oxidative stress or elevated homocysteine levels.

The compositions of the present invention include various vitamins, minerals, antioxidants and nutrients that improve the nutritional state of a patient; these compositions preferably may be used therapeutically or prophylacticly.

The vitamins, minerals, antioxidants and nutrients of the present invention may comprise carotenoids, vitamin E, vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein. In a specific embodiment, the compositions of the present invention may include more than 480 IU vitamin D. In another embodiment of the present invention, the compositions may comprise carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein. In another embodiment of the present invention, the compositions may further comprise one or more pharmaceutically acceptable carriers.

In another embodiment of the present invention, the compositions may comprise carotenoids selected from the group consisting of alpha-carotene, beta-carotene, cryptoxanthin, lutein, and zeaxanthin. In another embodiment of the present invention, the carotenoids may include lutein and zeaxanthin.

In another embodiment of the present invention, the compositions may include vitamin D, wherein vitamin D comprises vitamin D3. In another embodiment of the present invention, the compositions may include vitamin B12, wherein vitamin B12 comprises cobalamin. In another embodiment of the present invention, the compositions may include vitamin B6, wherein vitamin B6 comprises pyridoxine. In another embodiment of the present invention, the compositions may include vitamin B1, wherein vitamin B1 comprises thiamin. In another embodiment of the present invention, the compositions may include vitamin B2, wherein vitamin B2 comprises riboflavin. In another embodiment of the present invention, the compositions may include vitamin B3, wherein vitamin B3 comprises niacin. In another embodiment of the present invention, the compositions may include vitamin B5, wherein vitamin B5 comprises pantothenic acid.

In another embodiment of the present invention, the compositions may comprise vitamin D present in the range of about 500 IU to about 1500 IU. In another embodiment of the present invention, the compositions may comprise vitamin D present in the amount of about 1000 IU.

In another embodiment of the present invention, the compositions may comprise carotenoids, vitamin E, more than 480 IU vitamin D3, vitamin C, folic acid, pyridoxine, cobalamin, thiamin, riboflavin, niacin, pantothenic acid, biotin, magnesium, manganese, zinc, selenium, chromium, copper, alpha lipoic acid, and lutein. In another embodiment of the present invention, the compositions may comprise about 1500 IU to about 4500 IU carotenoids, about 50 IU to about 150 IU vitamin E, about 500 IU to about 1500 IU vitamin D3, about 150 mg to about 450 mg vitamin C, about 0.5 mg to about 1.5 mg folic acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein. In another embodiment of the present invention, the compositions may comprise about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, and about 5 mg lutein. In another embodiment of the present invention, the compositions may further comprise one or more pharnaceutical carriers and is administrable to a patient. In another embodiment of the present invention, the compositions may be administrable to the patient orally.

The present invention also includes methods of administering the compositions of the present invention to a patient for both prophylactic and therapeutic nutritional supplementation. In a specific embodiment of the present invention, the methods may include administering to a patient a composition comprising carotenoids, vitamin E, vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein. In a specific embodiment, the compositions of the present invention may include more than 480 IU vitamin D. In another embodiment of the present invention, the methods may utilize compositions comprising carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein. In another embodiment of the present invention, the compositions may further comprise one or more pharmaceutically acceptable carriers.

In another embodiment of the present invention, the methods may utilize compositions comprising carotenoids selected from the group consisting of alpha-carotene, beta-carotene, cryptoxanthin, lutein, and zeaxanthin. In another embodiment of the present invention, the carotenoids may include lutein and zeaxanthin.

In another embodiment of the present invention, the methods may utilize vitamin D, wherein vitamin D comprises vitamin D3. In another embodiment of the present invention, the methods may utilize vitamin B12, wherein vitamin B12 comprises cobalamin. In another embodiment of the present invention, the methods may utilize vitamin B6, wherein vitamin B6 comprises pyridoxine. In another embodiment of the present invention, the methods may utilize vitamin B1, wherein vitamin B1 comprises thiamin. In another embodiment of the present invention, the methods may utilize vitamin B2, wherein vitamin B2 comprises riboflavin. In another embodiment of the present invention, the methods may utilize vitamin B3, wherein vitamin B3 comprises niacin. In another embodiment of the present invention, the methods may utilize vitamin B5, wherein vitamin B5 comprises pantothenic acid.

In another embodiment of the present invention, the methods may utilize compositions comprising vitamin D present in the range of about 500 IU to about 1500 IU. In another embodiment of the present invention, the methods may utilize compositions comprising vitamin D present in the amount of about 1000 IU.

In another embodiment of the present invention, the methods may utilize compositions comprising carotenoids, vitamin E, more than 480 IU vitamin D3, vitamin C, folic acid, pyridoxine, cobalamin, thiamin, riboflavin, niacin, pantothenic acid, biotin, magnesium, manganese, zinc, selenium, chromium, copper, alpha lipoic acid, and lutein. In another embodiment of the present invention, the methods may utilize compositions comprising about 1500 IU to about 4500 IU carotenoids, about 50 IU to about 150 IU vitamin E, about 500 IU to about 1500 IU vitamin D3, about 150 mg to about 450 mg vitamin C, about 0.5 mg to about 1.5 mg folic acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein. In another embodiment of the present invention, the compositions may comprise about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 pig chromium, about 1.5 mg copper, about 15 mg alpha lipoic acid, and about 5 mg lutein. In another embodiment of the present invention, the methods may utilize compositions comprising one or more pharmaceutical carriers and are administrable to a patient. In another embodiment of the present invention, the methods may utilize compositions wherein the composition may be administered to the patient orally.

### DETAILED DESCRIPTION

It is understood that the present invention is not limited to the particular methodologies, protocols, fillers, excipients, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “a vitamin” is a reference to one or more vitamins and includes equivalents thereof known to those skilled in the art and so forth.

Unless defined otherwise, all technical and scientific terms used herein have the same meanings as commonly understood by one of ordinary skill in the art to which this invention belongs. Specific methods, devices, and materials are described, although any methods and materials similar or equivalent to those described herein can be used in the practice or testing of the present invention.

The term “subject,” as used herein, comprises any and all organisms and includes the term “patient.” “Subject” may refer to a human or any other animal.

The phrase “pharmaceutically acceptable,” as used herein, refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Thus, the phrase “pharmaceutically acceptable carriers,” as used herein, refers to such suitable compounds and materials defined above that may be added to the dosage form to assist in satisfactory processing of the dosage form or provide desirable physical characteristics to the dosage form. For example, “pharmaceutically acceptable carriers” may include, but is not limited to, binders, diluents, lubricants, glidants, colorants, emulsifiers, disintegrants, starches, water, oils, alcohols, preservatives, and sugars.

The phrase “swallowable form” refers to any compositions that do not readily dissolve when placed in the mouth and may be swallowed whole without any chewing or discomfort. Such compositions, in one embodiment, may have a shape containing no sharp edges and a smooth, uniform and substantially bubble free outer coating.

The term “dosage form,” as used herein, is the form in which the dose is to be administered to the subject or patient. The drug or supplement is generally administered as part of a formulation that includes nonmedical agents. The dosage form has unique physical and pharmaceutical characteristics. Dosage forms may be solid, liquid or gaseous. Solid forms include, but are not limited to pills, capsules, tablets, gel caplets, softgels, lozenges, wafers etc.

The term “substantially free of added” as used herein, means free from therapeutically effective amounts of compounds when administered in suggested doses, but may include trace amounts of compounds in non-therapeutically effective amounts.

As used herein, the terms “inactive,” “inert,” “excipient,” and/or “formulatory” refer to any compound that is an inactive ingredient of a described composition. The definition of “inactive ingredient” as used herein follows that of the U.S. Food and Drug Administration, as defined in 21 C.F.R. 201.3(b)(8), which is any component of a drug product other than the active ingredient.

By “active ingredient,” then, is meant any compound intended to furnish pharmacological activity or other direct effect in the diagnosis, cure, mitigation, treatment and/or prevention of a condition. See 21 C.F.R. 210.3(b)(7). Further, “active ingredients” include those compounds of the composition that may undergo chemical change during the manufacture of the composition and be present in the final composition in a modified form intended to furnish an activity or effect. Id.

The term “administrable” defines a composition that is able to be given to a patient. Likewise, “administering” refers to the act of giving a composition to a patient or otherwise making such composition available to a patient or the patient taking a composition.

As used herein, the term “about,” when located before a dosage amount or dosage range of a specific ingredient, refers to an amount or range closely above and/or closely below the stated amount or range that does not manifestly alter the therapeutic effect of the specific ingredient from the stated amount or range.

As used herein, the term “comprising” means that the present invention includes the listed ingredients thereafter, but does not exclude additional or unrecited ingredients.

As used herein, the term “consisting essentially of” means that the present invention includes the listed ingredients thereafter, and excludes only the ingredients that materially affect the basic and novel characteristic of the composition.

As used herein, the term “consisting of” means that the present invention includes only the ingredients listed thereafter.

Proper nutrition is essential for maintaining health and preventing diseases. The compositions and methods of the present invention provide the means to optimize good health by utilizing vitamin, mineral, and antioxidant nutritional supplementation. Specifically, the compositions of the present invention contain a variety of antioxidants, which may minimize free radical concentrations and minimize the deleterious effects of oxidative stress, and vitamins and minerals that support normal levels of homocysteine. More specifically, the compositions and methods of the present invention provide a beneficial increased supplementation of vitamin D due to the recent awareness of the increased role and importance that vitamin D has in the human body.

The compositions of the present invention may also comprise antioxidant factors that may protect against oxidative stress. Oxidative stress occurs as the body's natural use of oxygen creates unstable molecules known as free radicals, which steal stable electron partners from other molecules, launching more free radicals and increased molecular and cellular instability. This coxidative stress' is implicated in over 200 diseases, including cardiovascular disease and cancer, which are attributed to free radical oxidation. Rock et al., 96 J. AM. DIETARY ASSOC. 693-702 (1996). For example, lipid peroxidation is the initiating step in the oxidation of low-density lipoproteins (LDL). In turn, the lipid peroxides oxidate other lipoproteins, which are taken up by the cells of the arterial wall. Eventually, the deposited oxidized lipoproteins form an atherosclerotic plaque. Id. The antioxidant components of the compositions and methods described herein may preferably include one or more of vitamin E, selenium, vitamin C, carotenoids, lutein, and lipoic acid.

The compositions and methods of the present invention may also include B-complex vitamins, which are critical for health as each is part of one or more coenzymes in metabolizing food properly. This class of vitamins is water-soluble nutrients, not stored significantly in the body. Importantly, the B-complex vitamins may help normalize homocysteine levels and metabolism. High homocysteine levels have been correlated directly with increased risk of atherosclerosis and other heart disease. Although the exact mechanism by which homocysteine contributes to heart disease is not fully understood, it may act as an endothelial irritant that promotes atherosclerosis.by inducing endothelial dysfunction. B-complex vitamins are required for the proper function of the homocysteine metabolic pathway, thus maintaining adequate levels of these vitamins may assist in normalizing homocysteine levels and maintaining good health. The B-complex vitamins of the present compositions and methods may preferably include one or more of thiamin (B1), riboflavin (B2), niacin (B3), pantothenic acid (B5), biotin, folic acid, pyridoxine (B6) and cobalamin (B12).

Minerals are inorganic, or non-carbon-containing, elements that are critical for healthy physiological processes, and are contemplated in the compositions and methods of the present invention. For example, minerals act as cofactors for hundreds of enzymes that range from those associated, for example, with food digestion, nucleic acid production, protein synthesis to antioxidant enzymes. One particular mineral, chromium, is essential in healthy insulin function, as it plays a direct role in insulin's interactions at the cellular level. The minerals of the compositions and methods of the present invention may preferably include one or more of chromium, zinc, copper, magnesium, and manganese.

The compositions and methods of the present invention may also include vitamin D. Specifically, the compositions and methods of the present invention may include a beneficially increased supplementation of vitamin D. Vitamin D is a fat-soluble “hormone like” substance essential for healthy bones. This vitamin increases the absorption of calcium and phosphorous from the gastrointestinal tract, and improves essential mineral resorption into bone tissue. Vitamin D can be converted to its active form from exposure of the skin to sunlight. Clemens et al., 1 LANCET. 74-76 (1982); Maclaughlin et al., 76 J CLIN INVEST. 1536-39 (1985). This fact is among the reasons why vitamin D deficiency is common in the elderly, notably the institutionalized, who spend little or no time out of doors. Deficiencies lead to increased bone turnover and loss, and when severe, osteomalacia or softening of the bones. Supplementation with vitamin D has been shown to moderately reduce bone loss, increase serum 25-hydroxyvitamin D, and decrease serum parathyroid hormone levels. Dawson-Hughes et al., 337 NEW ENG. J. MED. 670-76 (1997); Lips et al., 86 J. CLIN. ENDOCRINOL. METAB. 1212-21 (2001).

Moreover, recent research suggests that vitamin D has more positive physiological effects than previous thought. Bischoff-Ferrari H A, 624 ADV EXP MED BIOL. 55-71 (2008); Holick M F, 357 N. ENG. J. MED. 266-81, (2007); Parikin et al., 89(3) J CLIN ENDOCRINOL METAB. 1196-99 (2004). For example, it has recently been determined that vitamin D also has a role in the enhancement of vascular function, defense against cancer, immuno-competence, blood pressure regulation and possessing the ability to enhance cellular insulin sensitivity in the human body. Due to the additional roles that vitamin D plays in the human body, it has recently been determined that higher daily vitamin D intake beyond current recommendations may be associated with better health outcomes. Bischoff-Ferrari HA, supra. Indeed, studies suggest increasing the serum level of 25-hydroxyvitamin D, a beneficial derivative of vitamin D, to a 30 ng/ml serum range. Id. A 30 ng/ml appears to be the most advantageous serum levels in recent studies reviewing patient bone mineral density (BMD), lower extremity function, dental health, risk of falls, admission to nursing home, fractures, cancer prevention and incident hypertension. Id.

Further, studies suggest that an intake of about 1000 IU of vitamin D3 (cholecalciferol) per day for all adults may bring at least 50% of the population up to the 30 ng/ml serum range for 25-hydroxyvitamin D. Id. Current nutritional supplements, however, do not provide a high enough dosage for obtaining such a high serum level of 25-hydroxyvitamin D. Presently, the suggested daily amount of vitamin D, as stated by the U.S. Dietary Reference Intake for adequate intake (AI) of vitamin D for infants, children and men and women aged 19-50 is 200 IU/day. Adequate intake increases to 400 IU/day for men and women aged 51-70 and up to 600 IU/day past the age of 70. Id. Due to these studies, present nutritional supplements may be insufficient to remedy the current U.S. and global epidemic related to vitamin D deficiency.

A nutritional supplement that includes a higher dosage amount of vitamin D in nutritional supplements is therefore currently needed. Thus, one embodiment of the present invention includes compositions and methods designed to optimize health and wellness, minimize oxidative stress, and provide a beneficial increased supplementation of vitamin D.

In one embodiment of the present invention, vitamin D is present in an amount more than 480 IU. In another embodiment of the invention, vitamin D is present in the amount ranging from about 500 IU to about 1500 IU. In another embodiment, vitamin D is present in the amount ranging from about 750 IU to about 1250 IU. In another embodiment, vitamin D is present in the amount ranging form about 900 IU to about 1100 IU.

Preferably, the vitamin D of the compositions and methods of the present invention is vitamin D3. In the body, vitamin D3 is produced when its precursor is exposed to ultraviolet irradiation (e.g., sunlight) and then hydroxylated in the liver to form 25-hydroxyvitamin D3, the major form of vitamin D in the circulation. This form of the vitamin may be hydroxylated again in the kidney, yielding 1,25 hydroxyvitamin D3, the most potent form of vitamin D. As noted above, vitamin D3 plays a role in the maintenance of calcium and phosphorus homeostasis, but it is also active in cell differentiation and immune function. In a particularly preferred embodiment, vitamin D3 is present in a range of about 500 IU to about 1500 IU. In another particularly preferred embodiment, vitamin D3 is present in an amount of about 1000 IU.

One specific embodiment of the present invention may include anti-oxidant factors, B-complex vitamins and other desirable vitamins and minerals. In a specific embodiment of the present invention, the compositions and methods may include carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein. In another specific embodiment of the present invention, the compositions and methods may comprise carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein. In another specific embodiment of the present invention, the compositions and methods may consist essentially of carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid and lutein.

In a specific embodiment, the compositions and methods of the present invention may include one or more pharmaceutically acceptable carriers. In a specific embodiment, a pharmaceutically acceptable carrier may be selected from one or more of the group consisting of binders, diluents, lubricants, glidants, colorants, emulsifiers, disintegrants, starches, water, oils, alcohols, preservatives and sugars. In another specific embodiment of the present invention, the compositions and methods may consist of carotenoids, vitamin E, more than 480 IU vitamin D, vitamin C, Colic acid, vitamin B6, vitamin B12, vitamin B1, vitamin B2, vitamin B3, vitamin B5, biotin, magnesium, manganese, zinc, selenium, chromium, copper, alpha lipoic acid, lutein and one or more pharmaceutically acceptable carriers.

As discussed previously, the antioxidant components of the compositions and methods described herein preferably include vitamin E, selenium, vitamin C, carotenoids, lutein, and lipoic acid.

Vitamin E is a fat-soluble vitamin antioxidant found in biological membranes where it protects the phospholipid membrane from oxidative stress. More specifically, alpha-tocopherol, the most abundant and most active form of the vitamin E family, is the principle lipid-soluble, chain breaking antioxidant in tissue and plasma. RECOMMENDED DIETARY ALLOWANCES 99-101 (Nat'l Research Council, 10th ed., 1989) (hereinafter “RDA”). Vitamin E inhibits the oxidation of unsaturated fatty acids by trapping peroxyl free radicals. It is also an antiatherogenic agent, and studies have demonstrated a reduced risk of coronary heart disease with increased intake of vitamin E. Stampfer et al., 328 New Eng. J. MED. 1444-49 (1993). Vitamin E is available in various forms known to those of skill in the art. In a specific embodiment of the present invention, vitamin E is present in an amount ranging from about 50 IU to about 150 IU. In another embodiment, vitamin E is present in an amount ranging from about 75 IU to about 125 IU. In another embodiment, vitamin E is present in an amount ranging from about 90 IU to about 110 IU. In another specific embodiment of the invention, vitamin E is present in an amount of about 100 IU. In another specific embodiment of the invention, vitamin E is present as d-alpha tocopheryl succinate. A specific embodiment of the invention includes about 100 IU d-alpha tocopheryl succinate.

Along with vitamin E, the mineral selenium is a component of the antioxidant enzyme, glutathione peroxidase, which plays a critical role in the control of oxygen metabolism, particularly catalyzing the breakdown of hydrogen peroxide. Burk, 3 ANN. REV. NUTRITION 53-70 (1983). Glutathione peroxidase prevents the generation of free radicals and decreases the risk of oxidative damage to numerous tissues, including the vascular system. Holben, 99 J. AM. DIETARY ASSOC. 836-43 (1999). Another selenoprotein is the enzyme iodothyronine 5′-diodinase that converts thyroxine (T₄) to triiodothyronine (T₃). Selenium is available in many forms known to those of ordinary skill in the art. In a preferred embodiment of the present invention, selenium is included in an amount ranging from about 50 μg to about 150 μg. In another embodiment, selenium is present in an amount ranging from about 75 μg to about 125 μg. In another embodiment, selenium is present in an amount ranging from about 90 μg to about 110 μg. In another specific embodiment of the invention, selenium is present in an amount of about 100 μg. In a specific embodiment of the invention, selenium is present as sodium selenate. A specific embodiment of the invention includes about 100 μg sodium selenate.

Vitamin C (also known as ascorbic acid) is another antioxidant present in the invention described herein. The major biochemical role of the water-soluble vitamin C is as a co-substrate in metal catalyzed hydroxylations, and it has antioxidant properties in interacting directly with superoxide hydroxyl radicals and singlet oxygen. Vitamin C also provides antioxidant protection for folate and vitamin E, keeping vitamin E in its most potent form. It also enhances the absorption of iron. RDA, at 115. In addition, vitamin C is required for collagen synthesis, epinephrine synthesis, and bile acid formation. Moreover, vitamin C has been implicated in inhibiting atherosclerosis by being present in extracellular fluid of the arterial wall and potentiating nitric oxide activity, thus normalizing vascular function. A specific embodiment of the compositions of the present invention includes a supplemental dose of vitamin C, preferably in the range of about 150 mg to about 450 mg. In another embodiment, vitamin C may be present in the range of about 225 mg to about 375 mg. In another embodiment of the present invention, vitamin C may be present in the range of about 270 mg to about 330 mg. A specific embodiment of the present invention includes vitamin C in the amount of about 300 mg.

Along with vitamins E and C, and selenium, carotenoids are a group of antioxidants embodied in the present invention. There are over 600 carotenoids occurring naturally in fruits and vegetables. Many of these fat-soluble compounds, of which beta-carotene is a well-known example, have pro-vitamin A activity as well as antioxidant activity. Less-known carotenoids include alpha-carotene, lutein, cryptoxanthine, and zeaxanthin. The compositions and methods herein include a cartonoid complex that closely mirrors that found naturally in fruits and vegetables. In particular, the carotenoids of the present invention may include alpha-carotene, beta-carotene, cryptoxanthin, lutein, and zeaxanthin. In particular, lutein and zeaxanthin are the major carotenoids that make up the macular pigment of the eye's retina, and their antioxidant properties protect the eye from light-induced. damage and macular degeneration. Berendschot et al., 41 INVEST. OPHTHALMOL. VIS. SCI. 3322-26 (2000). In a specific embodiment of the invention, carotenoids may be included in a range of about 1500 IU to about 4500 IU. In another embodiment of the invention, carotenoids may be present in a range of about 2250 IU to about 3750 IU. In another embodiment of the invention, carotenoids may be present in a range of about 2700 IU to about 3300 IU. In another embodiment of the invention, carotenoids may be present in an amount of about 3000 IU. In a more specific embodiment of the invention, the carotenoids include a mixture of alpha-carotene, beta-carotene, cryptoxanthin, lutein, and zeaxanthin. A specific embodiment of the invention includes about 3000 IU of a mixture of alpha-carotene, beta-carotene, cryptoxanthin, lutein, and zeaxanthin. In another specific embodiment, the carotenoids may include lutein and zeaxanthin.

Lutein is also preferably included in the compositions and methods described herein and is preferably included in an amount distinguished from that included in the mixed carotenoids. Regarding the antioxidant activity of lutein, scientists have demonstrated that lutein is an effective antioxidant capable of scavenging peroxyl radicals and quenching reactive oxygen species. Rapp et al., 41 INVEST. OPHTHALMOL. VIS. SCI. 1200-09 (2000). Thus, the compositions and methods of the present invention may include lutein, preferably in the amount ranging from about 2.5 mg to about 7.5 mg. In another embodiment, lutein may be present in the amount ranging from about 3.75 mg to about 6.25 mg. In another embodiment, lutein may be present in the amount ranging from about 4.5 mg to about 5.5 mg. A specific embodiment of the invention includes the amount of about 5 mg lutein.

Lipoic acid is an antioxidant and is preferably included in the compositions and methods of the present invention. Known as the “universal antioxidant,” alpha lipoic acid is both a lipid- and water-soluble antioxidant that works synergistically with other antioxidants in the cell's mitochondria. In addition to working with other antioxidant nutrients, lipoic acid has powerful, pro-antioxidant enzyme properties. Alpha lipoic acid is also a cofactor for several regulatory enzymes, including pyruvate dehydrogenase, and appears to have an effect on glucose transport and utilization. Rudich et al., 42 DIABETOLOGIA 949-57 (1999). Alpha lipoic acid also increases tocopherol activity and acts as a metal chelator. Furthermore, alpha lipoic acid improves microvascular perfusion. Haak et al., 108 EXPERIMENTAL & CLINICAL ENDOCRINOLOGY & DIABETES 168-74 (2000). In a specific embodiment of the compositions and methods of the present invention, alpha lipoic acid may be present in the amount ranging from about 7.5 mg to about 22.5 mg. In another embodiment, alpha lipoic acid may be present in the amount of about 18.75 mg to about 11.25 mg. In another embodiment, alpha lipoic acid may be present in the amount of about 13.5 mg to about 16.5 mg. A specific embodiment of the present invention may include alpha lipoic acid in the amount of about 15 mg.

In addition to antioxidants, the compositions and methods of the present invention also preferably include one or more B-complex vitamins such as thiamin (B1), riboflavin (B2), niacin (B3), pantothenic acid (B5), biotin, folic acid, pyridoxine (B6) and cobalamin (B12).

Thiamine (vitamin B1) plays a role in carbohydrate metabolism and neural function. It is a coenzyme for the oxidative decarboxylation of alpha-ketoacids (e.g., alpha-ketoglutarate and pyruvate) and for transketolase which is a component of the pentose phosphate pathway. Folate deficiency and malnutrition inhibit the activity of thiamine. RDA, at 123. One embodiment of the compositions of the present invention may include thiamin. In one embodiment, thiamin may be present in the amount ranging from about 10 mg to about 30 mg. In another embodiment, thiamin may be present in the amount ranging from about 15 mg to about 25 mg. In another embodiment, thiamin may be present in the amount ranging from about 18 mg to about 22 mg. In another embodiment, thiamin may be present in the amount of about 20 mg. In a specific embodiment of the present invention, the form of thiamine is thiamine HCl. Another specific embodiment of the invention comprises about 20 mg thiamine HCl.

Riboflavin (vitamin B2) is a component of two flavin coenzymes, flavin mononucleotide (FMN) and flavin adenine dinucleotide (FAD). These flavoenzymes are involved in a number of oxidation-reduction reactions including the conversion of pyridoxine and niacin. RDA, at 132. Flavoenzymes also play a role in a number of metabolic pathways such as citric acid cycle, amino acid deamination, purine degradation, and fatty acid oxidation and thus help to maintain carbohydrate, amino acid, and lipid metabolism. In one embodiment, the compositions and methods of the present invention may include riboflavin. In one embodiment, riboflavin may be present in the amount ranging from about 2.5 mg to about 7.5 mg. In another embodiment, riboflavin may be present in the amount ranging from about 3.75 mg to about 6.25 mg. In another embodiment, riboflavin may be present in the amount ranging from about 4.5 mg to about 5.5 mg. In another embodiment, riboflavin may be present in the amount of about 5 mg.

Niacin, also called vitamin B3, is the common name for two compounds: nicotinic acid (also called niacin) and niacinamide (also called nicotinamide). Niacin and is particularly important for maintaining healthy levels and types of fatty acids. Niacin is also required for the synthesis of pyroxidine, riboflavin, and folic acid. RDA, at 137. Administration of niacin may also produce a reduction in total cholesterol, LDL, and very low density lipoprotein (VLDL) levels; and an increase in high density lipoprotein (HDL) cholesterol levels. Nicotinamide adenine dinucleotide (NAD) and NAD phosphate (NADP) are active coenzymes of niacin. These coenzymes are involved in numerous enzymatic reactions such as glycolysis, fatty acid metabolism, and steroid synthesis. Henkin et al., 91 AM. J. MED. 239-46 (1991). One embodiment of the compositions and methods of the present invention may include niacin. In one embodiment, niacin may be present in the amount ranging from about 12.5 mg to about 37.5 mg. In another embodiment, niacin may be present in the amount ranging from about 18.75 mg to about 31.25 mg. In another embodiment, niacin may be present in the amount ranging from about 22.5 mg to about 27.5 mg. In another embodiment, niacin may be present in the amount of about 25 mg. In a preferred embodiment of the invention, niacin is present in the form of niacinamide. A specific embodiment of the invention includes about 25 mg of niacinamide.

Folic acid (vitamin B9), also called folate or methylfolate, is essential for the formation of red and white blood cells within bone marrow and also plays a role in heme formation. RDA, at 150. Folic acid in its active form, tetrahydrofolate, is a coenzyme that is involved in the transfer of methyl groups and it plays a role in DNA synthesis, purine synthesis, and amino acid synthesis, such as the conversion of glycine to serine and the transformation of homocysteine to methionine. The activation of folic acid requires a vitamin B12-dependent transmethylation and vitamin B12 is also necessary for folic acid delivery to tissues. Id. One embodiment of the compositions and methods of the present invention may comprise folic acid. In one embodiment of the present invention, folic acid may be present in the amount ranging from about 0.5 mg to about 1.5 mg. In another embodiment of the present invention, folic acid may be present in the amount ranging from about 0.75 mg to about 1.25 mg. In another embodiment of the present invention, folic acid may be present in the amount ranging from about 0.9 mg to about 1.1 mg. In another embodiment of the invention, folic acid may present in the amount of about 1 mg.

Pyridoxine (vitamin B6) is another B-complex vitamin included in the compositions and methods described herein. The administration of pyridoxine may reduce the levels of homocysteine. Bostom et al., 49 KIDNEY INT. 147-52 (1996). The active forms of pyridoxine, pyridoxal-5′-phosphate (PLP) and pyridoxamine-5′-phosphate, are coenzymes for numerous enzymes and as such, are essential for gluconeogenesis, niacin formation, and erythrocyte metabolism. RDA, at 142-143. Pyridoxine is a coenzyme for both cystathionine synthase and cystathionase, enzymes that catalyze the formation of cysteine from methionine. Homocysteine is an intermediate in this process and elevated levels of plasma homocysteine are recognized as a risk factor for vascular disease. Robinson et al., 94 CIRCULATION 2743-48 (1996). Hence, one embodiment of the compositions and methods of the present invention may include pyridoxine. In one embodiment of the present invention, pyridoxine may be present in the amount ranging from about 12.5 mg to about 37.5 mg. In another embodiment of the present invention, pyridoxine may be present in the amount ranging from about 18.75 mg to about 31.25 mg. In another embodiment of the present invention, pyridoxine may be present in the amount ranging from about 22.5 mg to about 27.5 mg. In another embodiment, pyridoxine may be present in the amount of about 25 mg. In a specific embodiment of the invention, pyridoxine is in the form of pyridoxine HCl. A specific embodiment of the invention includes about 25 mg pyridoxine HCl.

Biotin, another water-soluble B-complex vitamin, acts a coenzyme for a number of carboxylases, and thus has an important role in gluconeogenesis, fatty acid metabolism, and amino acid metabolism. RDA, at 166. For example, biotin serves as a carboxyl carrier for pyruvate carboxylase, which is involved in gluconeogenesis; acetyl CoA carboxylase, which is involved in fatty acid synthesis; and propionyl-CoA carboxylase, which is involved in glucose production. Researchers believe that biotin inhibits the effects of uremic toxins on tubulin polymerizaton. Braguer et al., 57 NEPHRON 192-96 (1991). Thus, one embodiment of the compositions and methods of the present invention may include biotin. In one embodiment of the present invention, biotin may be present in the amount ranging from about 50 μg to about 150 μg. In another embodiment of the present invention, biotin may be present in the amount ranging from about 75 μg to about 125 μg. In another embodiment of the present invention, biotin may be present in the amount ranging from about 90 μg to about 110 μg. In another embodiment of the present invention, biotin may be present in the amount of about 100 μg.

Pantothenic acid (vitamin B5) is a component of both the coenzyme A macromolecule and the acyl-carrier protein. These coenzymes function as carriers for acyl groups and are required for the synthesis of fatty acids, cholesterol, steroid hormones, and neurotransmitters. The coenzyme A complex also has a major role in the acetylation and acylation of numerous proteins. RDA, at 169. One embodiment of the compositions and methods of the present invention may include pantothenic acid. In one embodiment of the present invention, pantothenic acid may be present in the amount ranging from about 7.5 mg to about 22.5 mg. In another embodiment of the present invention, pantothenic acid may be present in the amount ranging from about 11.25 mg to about 18.75 mg. In another embodiment of the present invention, pantothenic acid may be present in the amount ranging from about 13.5 mg to about 16.5 mg. In another embodiment of the present invention, pantothenic acid may be present in the amount of about 15 mg. In a specific embodiment of the invention, pantothenic acid is present as calcium pantothenate. A specific embodiment of the invention includes about 15 mg calcium pantothenate.

Cobalamin (vitamin B12), another important vitamin included in the compositions and methods described herein, can be converted to the active coenzymes, methylcobalamin and 5′-deoxyadenosylcobalamin. These coenzymes are necessary for folic acid metabolism, conversion of coenzyme A, and myelin synthesis. For example, methylcobalamin catalyzes the demethylation of a folate cofactor, which is involved in DNA synthesis. A lack of demethylation may result in folic acid deficiency. RDA, at 159-160. Deoxyadenosylcobalamin is the coenzyme for the conversion of methylmalonyl-CoA to succinyl-CoA, which plays a role in the citric acid cycle. Importantly, cobalamin, along with pyridoxine and folic acid in implicated in the proper metabolism of homocysteine. Cobalamin is available as cyanocobalamin, methylcobalamin, hydroxocobalamin, adenosylcobalamin, and hydroxycyanocobalamin. One embodiment of the compositions and methods of the present invention may include cobalamin. In one embodiment of the present invention, cobalamin may be present in the amount ranging from about 25 μg to about 75 μg. In another embodiment, cobalamin may be present in the amount ranging from about 37.5 μg to about 62.5 μg. In another embodiment, cobalamin may be present in the amount ranging from about 45 μg to about 55 μg. In one embodiment of the present invention, cobalamin may be present in the amount of about 50 μg. In a specific embodiment of the invention, cobalamin is present as cyanocobalamin. Another specific embodiment of the invention includes about 50 μg cyanocobalamin.

As noted previously, minerals are inorganic elements that play a crucial role in physiological processes in the body relating to good health. The compositions and methods of the present invention may comprise minerals, and, in a preferred embodiment, comprise one or more of selenium, discussed above, and magnesium, manganese, zinc, chromium, and copper.

Magnesium is found primarily in both bone and muscle. Magnesium is an essential component for over 300 enzymes, including enzymes of biosynthetic pathways, glycolysis, protein synthesis, transketolase reactions, and membrane transport. Magnesium is also involved in the formation of cAMP, a cytosolic second messenger that plays a role in cell signaling mechanisms. In addition, magnesium functions both synergistically and antagonistically with calcium in neuromuscular transmission. RDA, at 188. Specifically, magnesium is critical for the maintenance of electrochemical potentials of nerve and muscle membranes and the neuromuscular junction transmissions, particularly important in the heart. Not surprisingly, magnesium deficiency is tied to cardiovascular disease and hypertension. Agus et al., 17 CRIT. CARE CLINICS 175-87 (2001). Indeed, oral magnesium therapy improves endothelial function in patients with coronary disease. Shechter et al., 102 CIRCULATION 2353-58 (2000). Yet, most individuals in the U.S. receive only about seventy-five percent of the magnesium they need from their diets. Magnesium is available in a variety of salts. One embodiment of the compositions and methods of the present invention may include magnesium. In one embodiment of the present invention, magnesium may be present in the amount ranging from about 25 mg to about 75 mg. In another embodiment of the present invention, magnesium may be present in the amount ranging from about 37.5 mg to about 62.5 mg. In another embodiment of the present invention, magnesium may be present in the amount ranging from about 45 mg to about 55 mg. In one embodiment of the present invention, magnesium may be present in the amount of about 50 mg. In a specific embodiment of the invention, magnesium may be present as magnesium oxide. A specific embodiment of the invention includes about 50 mg magnesium oxide.

Manganese, like magnesium, plays a key role in multiple enzymes and is needed for healthy skin, bone, and cartilage formation, as well as glucose tolerance. For example, manganese is a cofactor for enzymes such as glutamine synthetase, pyruvate carboxylase, and mitochrondrial superoxide dismutase. RDA, at 230. In particular, manganese is essential for glycoprotein and proteoglycan synthesis, and thus is involved in the formation of connective and skeletal tissue, as well as carbohydrate and lipid metabolism. It also helps activate superoxide dismutase, an important antioxidant enzyme. Manganese is available in many forms known to those of ordinary skill in the art, including manganese sulfate, manganese oxide, manganese oxy-sulfate, and manganese proteinate. One embodiment of the compositions and methods of the present invention may include manganese. In one embodiment of the present invention, manganese may be present in the amount ranging from about 0.75 mg to about 2.25 mg. In another embodiment of the present invention, manganese may be present in the amount ranging from about 1.125 mg to about 1.875 mg. In another embodiment of the present invention, manganese may be present in the amount ranging from about 1.35 mg to about 1.65 mg. In one embodiment of the present invention, manganese may be present in the amount of about 1.5 mg. In a specific embodiment, manganese is present as manganese sulfate. A specific embodiment of the invention includes about 1.5 mg of manganese sulfate.

Zinc plays a role in numerous metabolic activities such as nucleic acid production, protein synthesis, and development of the immune system. There are more than 200 zinc metalloenzymes including aldolase, alcohol dehydrogenase, RNA polymerase, and protein kinase C. Zima et al., 17 BLOOD PURIF. 182-86 (1999). Moreover, zinc stabilizes RNA and DNA structures, forms zinc fingers in nuclear receptors, and is a component of chromatin proteins involved in transcription and replication. Zinc is available in many forms, such as zinc oxide and zinc sulfate. One embodiment of the compositions and methods of the present invention may include zinc. In one embodiment of the present invention, zinc may be present in the amount ranging from about 12.5 mg to about 37.5 mg. In another embodiment of the present invention, zinc may be present in the amount ranging from about 18.75 mg to about 31.25 mg. In one embodiment of the present invention, zinc may be present in the amount of about 25 mg. In a specific embodiment, zinc may be present as zinc oxide. Another specific embodiment of the present invention includes about 25 mg zinc oxide.

The trace mineral chromium harmonizes with insulin at the cellular level to optimize the release of energy from glucose, as well as maintaining proper cellular lipid or fat metabolism. Specifically, chromium increases insulin binding to cells, insulin receptor number, and activates the insulin receptor kinase leading to increased insulin sensitivity. Several studies suggest that adequate chromium levels are needed for optimal glycemic control. See, e.g., Anderson et al., 26 DIABETES METABOLABOLISM 22-27 (2000); Vincent, 130 J. NUTRITION 715-18 (2000). The concentration of chromium declines with age, and coronary artery disease appears to be associated with low levels of chromium. RDA, at 241. Yet, ninety percent of adults in the U.S. consume less than the recommended minimum amount of chromium. Chromium is available in various forms known to those skilled in the art, such as chromium chloride, chromium sulfate, chromium potassium sulfate, and chromium picolinate. One embodiment of the compositions and methods of the present invention may include chromium. In one embodiment of the present invention, chromium may be present in the amount ranging from about 25 μg to about 75 μg. In another embodiment of the present invention, chromium may be present in the amount ranging from about 37.5 μg to about 62.5 μg. In another embodiment of the present invention, chromium may be present in the amount ranging from about 45 μg to about 55 μg. In one embodiment of the present invention, chromium may be present in the amount of about 50 μg. Specifically, chromium may be supplied as chromium chloride. A specific embodiment of the invention includes about 50 μg chromium chloride.

Copper is a component of several enzymes associated with numerous physiological functions, including, for example, oxidase enzymes, such as cytochrome c oxidase, and cytosolic superoxide dismutase. RDA, at 224. In particular, copper is a cofactor of lysyl oxidase, which is critical for lysine cross-linking in collagen and elastin. Copper acts as an antioxidant, and promotes the synthesis of melanin and catecholamines. In addition, copper is present in the blood as ceruloplasmin which is involved in oxidizing iron prior to transport to the plasma. Copper is available in multiple forms, such as cupric oxide, copper sulfate, cupric acetate, and alkaline copper carbonate. One embodiment of the compositions and methods of the present invention may include copper. In one embodiment of the present invention, copper may be present in the amount ranging from about 0.75 mg to about 2.25 mg. In another embodiment of the present invention, copper may be present in the amount ranging from about 1.125 mg to about 1.875 mg. In another embodiment of the present invention, copper may be present in the amount ranging from about 1.35 mg to about 1.65 mg. In one embodiment of the present invention, copper may be present in the amount of about 1.5 mg. In a specific embodiment of the invention, copper may be present as cupric sulfate. A specific embodiment includes about 1.5 mg cupric sulfate.

The composition and methods may include various combinations of vitamins, minerals, nutrients and antioxidants comprising specific forms listed above. For example, the compositions and methods of the present invention may include carotenoids, vitamin E, vitamin D3, vitamin C, folic acid, pyridoxine, cobalamin, thiamin, riboflavin, niacin, pantothenic acid, biotin, magnesium, manganese, zinc, selenium, chromium, copper, alpha lipoic acid, and lutein. In another specific embodiment, the compositions and methods of the present invention may comprise carotenoids, vitamin E, vitamin D3, vitamin C, folic acid, pyridoxine, cobalamin, thiamin, riboflavin, niacin, pantothenic acid, biotin, magnesium, manganese, zinc, selenium, chromium, copper, alpha lipoic acid, and lutein. In another specific embodiment, the compositions and methods of the present invention may consist essentially of carotenoids, vitamin E, vitamin D3, vitamin C, folic acid, pyridoxine, cobalamin, thiamin, riboflavin, niacin, pantothenic acid, biotin, magnesium, manganese, zinc, selenium, chromium, copper, alpha lipoic acid, and lutein. In another specific embodiment, the compositions and methods of the present invention may consist of carotenoids, vitamin E, vitamin D3, vitamin C, folic acid, pyridoxine, cobalamin, thiamin, riboflavin, niacin, pantothenic acid, biotin, magnesium, manganese, zinc, selenium, chromium, copper, alpha lipoic acid lutein and one or more pharmaceutically acceptable carriers. In a specific embodiment, the amount of vitamin D in these methods and compositions may be more than 480 IU.

In another embodiment, the present invention may include compositions and methods of ranges or amounts of the vitamins, minerals, nutrients and antioxidants. In another specific embodiment, the compositions and methods of the present invention may include about 1500 IU to about 4500 IU carotenoids, about 50 IU to about 150 IU vitamin E, about 500 IU to about 1500 IU vitamin D3, about 150 mg to about 450 mg vitamin C, about 0.5 mg to about 1.5 mg folio acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein. In another specific embodiment, the compositions and methods of the present invention may comprise about 1500 IU to about 4500 IU carotenoids, about 50 IU to about 150 IU vitamin E, about 500 IU to about 1500 IU vitamin D3, about 150 mg to about 450 mg vitamin C, about 0.5 mg to about 1.5 mg folio acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein. In another specific embodiment, the compositions and methods of the present invention may consist essentially of about 1500 IU to about 4500 IU carotenoids, about 50 IU to about 150 IU vitamin E, about 500 IU to about 1500 IU vitamin D3, about 150 mg to about 450 mg vitamin C, about 0.5 mg to about 1.5 mg folic acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein. In another specific embodiment, the compositions and methods of the present invention may consist of about 1500 IU to about 4500 IU carotenoids, about 50 IU to about 150 IU vitamin E, about 500 IU to about 1500 IU vitamin D3, about 150 mg to about 450 mg vitamin C, about 0.5 mg to about 1.5 mg folic acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, about 2.5 mg to about 7.5 mg lutein and one or more pharmaceutically acceptable carriers.

In another specific embodiment, the compositions and methods of the present invention may include about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, and about 5 mg lutein. In another specific embodiment, the compositions and methods of the present invention may comprise about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, and about 5 mg lutein. In another specific embodiment, the compositions and methods of the present invention may consist essentially of about 3000 IU carotenoids, about 100 IU vitamin E, about 1000 IU vitamin D3, about 300 mg vitamin C, about 1.0 mg folic acid, about 25 mg pyridoxine, about 50 pLg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, and about 5 mg lutein. In another specific embodiment, the compositions and methods of the present invention may consist of about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, about 5 mg lutein and one or more pharmaceutically acceptable carriers.

In another embodiment of the present invention, the compositions and methods may include carotenoids, vitamin E, vitamin D3, vitamin C (Ascorbic Acid), thiamine HCl, riboflavin, niacinamide, folic acid, pyridoxine HCl, biotin, calcium pantothenate, cyanocobalamin, magnesium oxide, manganese sulfate, zinc oxide, sodium selenate, chromium chloride, cupric sulfate, alpha lipoic acid and lutein. In another embodiment of the present invention, the compositions and methods may comprise carotenoids, vitamin E, vitamin B3, vitamin C (Ascorbic Acid), thiamine HCl, riboflavin, niacinamide, folic acid, pyridoxine HCl, biotin, calcium pantothenate, cyanocobalamin, magnesium oxide, manganese sulfate, zinc oxide, sodium selenate, chromium chloride, cupric sulfate, alpha lipoic acid and lutein. In another embodiment of the present invention, the compositions and methods may consist essentially of carotenoids, vitamin E, vitamin D3, vitamin C (Ascorbic Acid), thiamine HCl, riboflavin, niacinamide, folic acid, pyridoxine HCl, biotin, calcium pantothenate, cyanocobalamin, magnesium oxide, manganese sulfate, zinc oxide, sodium selenate, chromium chloride, cupric sulfate, alpha lipoic acid and lutein. In another embodiment of the present invention, the compositions and methods may consist of carotenoids, vitamin E, vitamin D3, vitamin C (Ascorbic Acid), thiamine HCl, riboflavin, niacinamide, folic acid, pyridoxine HCl, biotin, calcium pantothenate, cyanocobalamin, magnesium oxide, manganese sulfate, zinc oxide, sodium selenate, chromium chloride, cupric sulfate, alpha lipoic acid, lutein and one or more pharmaceutically acceptable carriers.

In another embodiment of the present invention, the compositions and methods may include about 3000 IU carotenoids, about 100 IU vitamin E, about 1000 IU vitamin D3, about 300 mg vitamin C (Ascorbic Acid), about 20 mg thiamine HCl, about 5 mg riboflavin, about 25 mg niacinamide, about I mg folic acid, about 25 mg pyridoxine HCl, about 100 μg biotin, about 15 mg calcium pantothenate, about 50 μg cyanocobalamin, about 50 mg magnesium oxide, about 1.5 mg manganese sulfate, about 25 mg zinc oxide, about 100 μg sodium selenate, about 50 μg chromium chloride, about 1.5 mg cupric sulfate, about 15 mg alpha lipoic acid and about 5 mg lutein. In another embodiment of the present invention, the compositions and methods may comprise about 3000 IU carotenoids, about 100 IU vitamin E, about 1000 IU vitamin D3, about 300 mg vitamin C (Ascorbic Acid), about 20 mg thiamine HCl, about 5 mg riboflavin, about 25 mg niacinamide, about 1 mg folic acid, about 25 mg pyridoxine HCl, about 100 μg biotin, about 15 mg calcium pantothenate, about 50 μg cyanocobalamin, about 50 mg magnesium oxide, about 1.5 mg manganese sulfate, about 25 mg zinc oxide, about 100 μg sodium selenate, about 50 μg chromium chloride, about 1.5 mg cupric sulfate, about 15 mg alpha lipoic acid and about 5 mg lutein. In another embodiment of the present invention, the compositions and methods may consist essentially of about 3000 IU carotenoids, about 100 IU vitamin E, about 1000 IU vitamin D3, about 300 mg vitamin C (Ascorbic Acid), about 20 mg thiamine HCl, about 5 mg riboflavin, about 25 mg niacinamide, about 1 mg folic acid, about 25 mg pyridoxine HCl, about 100 μg biotin, about 15 mg calcium pantothenate, about 50 μg cyanocobalamin, about 50 mg magnesium oxide, about 1.5 mg manganese sulfate, about 25 mg zinc oxide, about 100 μg sodium selenate, about 50 μg chromium chloride, about 1.5 mg cupric sulfate, about 15 mg alpha lipoic acid and about 5 mg lutein. In another embodiment of the present invention, the compositions and methods may consist of about 3000 IU carotenoids, about 100 IU vitamin E, about 1000 IU vitamin D3 about 300 mg vitamin C (Ascorbic Acid), about 20 mg thiamine HCl, about 5 mg riboflavin, about 25 mg niacinamide, about 1 mg folic acid, about 25 mg pyridoxine HCl, about 100 μg biotin, about 15 mg calcium pantothenate, about 50 μg cyanocobalamin, about 50 mg magnesium oxide, about 1.5 mg manganese sulfate, about 25 mg zinc oxide, about 100 μg sodium selenate, about 50 μg chromium chloride, about 1.5 mg cupric sulfate, about 15 mg alpha lipoic acid, about 5 mg stein and one or more pharmaceutically acceptable carriers.

The compositions and methods of the present invention represent a combination of essential vitamins and minerals that work together with various metabolic systems and physiological responses of the human body. The active ingredients are available from numerous commercial sources, and in several active forms or salts thereof, known to those of ordinary skill in the art. Hence, the compositions and methods of the present invention are not limited to any particular form of the vitamin or mineral ingredient described herein.

While supplementation with certain vitamins, antioxidants, nutrients and minerals protects against the onset of these diseases, other vitamins and minerals have been found to inhibit the beneficial effects of these certain vitamins, antioxidants, minerals and other nutrients. Thus, in a specific embodiment, the compositions and methods of the present invention may be substantially free of other added vitamins, antioxidants, minerals and nutrients. In a more specific embodiment, the compositions and methods of the present invention may be substantially free of all other added vitamins, minerals and nutrients except for carotenoids, vitamin E, vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, lutein and their equivalents.

B-complex vitamins, such as vitamins B6 and B12, vitamin D3, and magnesium play integral roles in physiological mechanisms that serve to prevent, treat and/or alleviate the occurrence or negative effects of cardiovascular disease, colorectal cancer and osteoporosis. Supplementation with vitamins and minerals such as vitamin A, vitamin K, and iron may inhibit these beneficial effects. Thus, when creating or choosing a nutritional supplement, it is essential to understand the physiological needs and risks of individual patients and population groups and the interactions between various vitamins and minerals.

For example, vitamin A may inhibit the beneficial effects of the present invention. Supplementation with vitamins and minerals such as vitamin A, may inhibit the beneficial effects of the B-complex vitamins, vitamin D₃, and magnesium. Moreover, elevated serum levels of the active form of vitamin A (retinol) are correlated with increased bone fragility with a resulting deleterious effect on bone health. Although retinol is involved in bone remodeling, excessive intake, as can occur with long term supplementation, has been linked to bone demineralization. Michaelson et al., 348(4) N. ENG. J. MED. 287-94 (2003); Feskanich et al., 287(1) JAMA 47-54 (2002). In a specific embodiment, the compositions and methods of the present invention may be substantially free of added vitamin A.

Another example of a vitamin that may have deleterious effects is vitamin K. Although vitamin K, or phylloquinone, plays a role in the process of maintaining bone health, it also plays a major role in the synthesis of coagulation factors. This delicate balance of coagulation is at times purposefully altered in those with, or at high risk of, cardiovascular disease. Increased intake of vitamin K can alter the efficacy of specific medications used for this purpose. Further, the human body produces vitamin K from naturally occurring intestinal bacteria, thus making deficiency of this nutrient rare. Due to these factors, broad spectrum vitamin K supplementation is discouraged. Kumik et al., 37(11) ANN. PHARMACOTHER. 1603-06(2003); Shearer, 345 LANCET 229-34 (1995). In a specific embodiment, the compositions and methods of the present invention may be substantially free of added vitamin K.

The mineral iron may be specifically excluded from the compositions and methods of the present invention. Although iron is an essential mineral with many functions, iron has also been implicated as a catalyst for lipid oxidation. Specifically, lipid oxidation associated with LDL cholesterol has been correlated strongly with an increased risk of cardiovascular disease. Moreover, some older patients exhibit sensitivity to iron or build-up of iron concentration in the liver. Although iron supplementation may be indicated in specific population groups or disease states, universal supplementation may not always be recommended. Hence, the compositions and methods of the present invention may be substantially free of added iron.

In another example, Lactose is a disaccharide, or sugar that is found mainly in milk and dairy products. Lactose intolerance or the inability to properly digest and absorb this compound is relatively common. With this inability comes uncomfortable side effects such as abdominal bloating, pain, and diarrhea upon ingestion of lactose-containing foods. Since milk and dairy products are a primary source of both calcium and lactose, those who are lactose intolerant are more likely to have insufficient calcium intake and therefore osteoporosis. DiStefano et al., 122(7) GASTROENTEROL. 1793-99 (2002). In a specific embodiment, the compositions and methods of the present invention may be free of added lactose.

Specific patients may also have unique or rare adverse reactions to antioxidants, vitamins or minerals added in the compositions of the present invention. Thus, in particular instances, the compositions and methods of the present invention may also be substantially free of one or more of the added specific antioxidants, vitamins, nutrients or minerals in the present invention. In one embodiment, the compositions and methods of the present invention may be substantially fee of added vitamin E. In one embodiment, the compositions and methods of the present invention may be substantially free of added vitamin C. In one embodiment, the compositions and methods of the present invention may be substantially free of added folic acid. In one embodiment, the compositions and methods of the present invention may be substantially free of added vitamin B6. In one embodiment, the compositions and methods of the present invention may be substantially free of added vitamin B12. In one embodiment, the compositions and methods of the present invention may be substantially free of added vitamin B1. In one embodiment, the compositions and methods of the present invention may be substantially free of added vitamin B2. In one embodiment, the compositions and methods of the present invention may be substantially free of added vitamin B3. In one embodiment, the compositions and methods of the present invention may be substantially free of added vitamin B5. In one embodiment, the compositions and methods of the present invention may be substantially free of added biotin. In one embodiment, the compositions and methods of the present invention may be substantially free of added magnesium. In one embodiment, the compositions and methods of the present invention may be substantially free of added manganese. In one embodiment, the compositions and methods of the present invention may be substantially free of added zinc. In one embodiment, the compositions and methods of the present invention may be substantially free of added selenium. In one embodiment, the compositions and methods of the present invention may be substantially free of added chromium. In one embodiment, the compositions and methods of the present invention may be substantially fee of added copper. In one embodiment, the compositions and methods of the present invention may be substantially free of added alpha lipoic acid. In one embodiment, the compositions and methods of the present invention may be substantially free of added lutein.

In another specific embodiment, the compositions and methods of the present invention may be in the form of a kit. In another specific embodiment, the kits may be utilized for various packaging of the composition. For example, in a specific embodiment, the present invention may comprise swallowable compositions packaged in blister packs. Blister packs as packaging for swollowable compositions are well known to those of ordinary skill in the art. Blister packs may be made of a transparent plastic sheet which as been formed to carry a matrix of depression or blisters. One or more swollowable compositions are received in each depression or blister. A foil or plastic backing is then adhered across the plane of the sheet seating the swollowable compositions in their respective blisters. Examples of materials used for the blister packs include, but are not limited to, aluminum, paper, polyester, PVC, and polypropylene. Alternative materials are known to those of ordinary skill in the art. To remove a swollowable composition, the depression material is pressed in and the composition is pushed through the backing material. Multiple blister packs may be placed in an outer package, often a box or carton for sale and distribution.

Another specific embodiment of the present invention may comprise swallowable compositions packaged in bottles. The bottle may be glass or plastic in form with a pop or screw top cap. Bottle packaging for compositions in swollowable form are well known to those of ordinary skill in the art.

Additionally, the unit dose forms may be individually wrapped, packaged as multiple units on paper strips or in vials of any size, without limitation. The swallowable, chewable or dissolvable compositions of the invention may be packaged in unit dose, rolls, bulk bottles, blister packs and combinations thereof, without limitation.

The ingredients of the present invention are preferably combined into a composition which may be in the form of a solid powder, caplets, tablets, lozenges, pills, capsules, softgel or a liquid, and which may be administered alone or in suitable combination with other components. For example, the composition of the present invention may be administered in one or more caplets or lozenges as practical for ease of administration. Each of the vitamins and minerals is commercially available, and can be blended to form a single composition or can form multiple compositions, which may be co-administered.

To prepare the compositions of the present invention, each of the active ingredients may be combined in intimate admixture with a suitable carrier according to conventional compounding techniques. The carrier may take a wide variety of forms depending upon the form of the preparation desired for administration, e.g., oral, sublingual, nasal, topical patch, or parenteral.

In preparing the composition in oral dosage form, any of the usual media may be utilized. For liquid preparations (e.g., suspensions, elixirs, and solutions), media containing, for example water, oils, alcohols, flavoring agents, preservatives, coloring agents and the like may be used. Pharmaceutical acceptable carriers such as starches, sugars, diluents, granulating agents, lubricants, binders, disintegrating agents and the like may be used to prepare oral solids (e.g., powders, caplets, pills, tablets, capsules, and lozenges). Controlled release forms may also be used. Because of their ease in administration, caplets, tablets, pills, and capsules represent the most advantageous oral dosage unit form, in which case solid carriers are employed. If desired, tablets may be sugar coated or enteric coated by standard techniques. All of these pharmaceutical carriers and formulations are well known to those of ordinary skill in the art. See, e.g., WADE & WALLER, HANDBOOK OF PHARMACEUTICAL EXCIPIENTS (2nd ed. 1994).

In a specific embodiment of the present invention may comprise compositions in the dosage form of a soft-gel. A soft-gel is a one-piece, sealed, soft gelatin shell that contains a solution, a suspension, or a semi-solid paste. Soft-gels are predominantly used to contain liquids wherein the active ingredients are present in the dissolved or suspended state. Soft-gels have been widely known and used for many years and for a variety of purposes. Because soft-gels have properties that are quite different from two-piece, hard shell capsules, the soft-gels are capable of retaining a liquid fill material. Soft-gels are often used to encapsulate consumable materials, including vitamins, dietary supplements, pharmaceuticals, and the like, in a liquid vehicle or carrier. Soft-gels arc a unique dosage form that can provide distinct advantages over more traditional dosage forms such as tablets, hard-shell capsules, and liquids. These advantages include patient compliance and consumer preference, improved bioavailability, speed of product development in many cases, shortened manufacturing time, enhanced drug stability due to less exposure of the active ingredient to oxygen, excellent dose uniformity, and product differentiation.

A specific embodiment of the present invention may comprise swallowable compositions. Swallowable compositions are well known in the art and are those that do not readily dissolve when placed in the mouth and may be swallowed whole without any chewing or discomfort. In a specific embodiment of the present invention the swallowable compositions may have a shape containing no sharp edges and a smooth, uniform and substantially bubble free outer coating.

In a specific embodiment of the swallowable compositions of the present invention, the surface of the compositions may be coated with a polymeric film. Such a film coating has several beneficial effects. First, it reduces the adhesion of the compositions to the inner surface of the mouth, thereby increasing the patient's ability to swallow the compositions. Second, the film may aid in masking the unpleasant taste of certain drugs. Third, the film coating may protect the compositions of the present invention from atmospheric degradation. Polymeric films that may be used in preparing the swallowable compositions of the present invention include vinyl polymers such as polyvinyl pyrrolidone, polyvinyl alcohol and acetate, cellulosics such as methyl and ethyl cellulose, hydroxyethyl cellulose and hydroxylpropyl methylcellulose, acrylates and methacrylates, copolymers such as the vinyl-maleic acid and styrene-maleic acid types, and natural gums and resins such as zein, gelatin, shellac and acacia.

In a specific embodiment of the present invention, the compositions may comprise chewable compositions. Chewable compositions are those that have a palatable taste and mouthfeel, are relatively soft and quickly break into smaller pieces and begin to dissolve after chewing such that they are swallowed substantially as a solution.

In order to create chewable compositions, certain ingredients should be included to achieve the attributes just described. For example, chewable compositions should include ingredients that create pleasant flavor and mouthfeel and promote relative softness and dissolvability in the mouth. The following discussion describes ingredients that may help to achieve these characteristics.

In one embodiment, the compositions and methods of the present invention may have a pleasant or palatable flavor. Palatable flavors may be achieved by including sweetening agents and/or flavorants. Sweetening agents that may be included in the compositions of the present invention include, by way of example and without limitation, sucrose, fructose, high fructose corn syrup, dextrose, saccharin sodium, maltodextrin, aspartame, potassium acesulfame, neohesperidin dihydrochalcone, sucralose, monoammonium glycyrrhizinate, and others known to those of ordinary skill in the art. As used herein, the term “flavorant” means natural or artificial compounds used to impart a pleasant flavor and often odor to a pharmaceutical preparation. Flavorants that may be used in the present invention include, for example and without limitation, natural and synthetic flavor oils, flavoring aromatics, extracts from plants, leaves, flowers, and fruits and combinations thereof. Such flavorants include, by way of example and without limitation, anise oil, cinnamon oil, vanilla, vanillin, cocoa, chocolate, natural chocolate flavor, menthol, grape, peppermint oil, oil of wintergreen, clove oil, bay oil, anise oil, eucalyptus, thyme oil, cedar leave oil, oil of nutmeg, oil of sage, oil of bitter almonds, cassia oil; citrus oils, such as lemon, orange, lime and grapefruit oils; and fruit essences, including apple, pear, peach, berry, wildberry, date, blueberry, kiwi, strawberry, raspberry, cherry, plum, pineapple, and apricot. All of these flavorants are commercially available. In a specific embodiment of the present invention, flavorants that may be used include natural berry extracts and natural mixed berry flavor, as well as citric and malic acid. The amount of flavorants used may depend on a number of factors, including desired taste characteristics. While not necessary, one or more of these sweetening agents and/or flavorants also may be included in the chewable and swallowable compositions of the present invention.

In addition to having a palatable flavor, chewable compositions also should have a pleasant mouthfeel. A variety of ingredients can be included in the compositions of the present invention to enhance mouthfeel.

In the chewable compositions of the present invention, sugars such as white sugar, corn syrup, sorbitol (solution), maltitol (syrup), oligosaccharide, isomaltooligosaccharide, sucrose, fructose, lactose, glucose, lycasin, xylitol, lactitol, erythritol, mannitol, isomaltose, dextrose, polydextrose, dextrin, compressible cellulose, compressible honey, compressible molasses and mixtures thereof may be added to improve mouthfeel and palatability. Further, by way of example and without limitation, fondant or gums such as gelatin, agar, arabic gum, guar gum, and carrageenan may be added to improve the chewiness of the compositions. Fatty materials that may be included in the present invention include, by way of example and without limitation, vegetable oils (including palm oil, palm hydrogenated oil, corn germ hydrogenated oil, castor hydrogenated oil, cotton-seed oil, olive oil, peanut oil, palm olein oil, and palm stearin oil), animal oils (including refined oil and refined lard whose melting point ranges from 30° to 42° C.), Cacao fat, margarine, butter, and shortening.

Alkyl polysiloxanes (commercially available polymers sold in a variety of molecular weight ranges and with a variety of different substitution patterns) also may be used in the present invention to enhance the texture, the mouthfeel, or both of the chewable nutritional supplement compositions described herein. By “enhance the texture” it is meant that the alkyl polysiloxane improves one or more of the stiffness, the brittleness, and the chewiness of the chewable supplement, relative to the same preparation lacking the alkyl polysitoxane. By “enhance the mouthfeel” it is meant that the alkyl polysiloxane reduces the gritty texture of the supplement once it has liquefied in the mouth, relative to the same preparation lacking the alkyl polysiloxane.

Alkyl polysiloxanes generally comprise a silicon and oxygen-containing polymeric backbone with one or more alkyl groups pending from the silicon atoms of the back bone. Depending upon their grade, they can further comprise silica gel. Alkyl polysiloxanes are generally viscous oils. Exemplary alkyl polysiloxanes that can be used in the swallowable, chewable or dissolvable compositions of the present invention include, by way of example and without limitation, monoalkyl or dialkyl polysiloxanes, wherein the alkyl group is independently selected at each occurrence from a C₁-C₆-alkyl group optionally substituted with a phenyl group. A specific alkyl polysiloxane that may be used is dimethyl polysiloxane (generally referred to as simethicone). More specifically, a granular simethicone preparation designated simethicone GS may be used. Simethicone GS is a preparation which contains 30% simethicone USP. Simethicone USP contains not less than about 90.5% by weight (CH₃)₃—Si{OSi(CH₃)₂}CH₃ in admixture with about 4.0% to about 7.0% by weight SiO₂.

To prevent the stickiness that can appear in conventional chewable compositions and to facilitate conversion of the active ingredients to emulsion or suspension upon taking, the compositions of the present invention, may further comprise emulsifiers such as, by way of example and without limitation, glycerin fatty acid ester, sorbitan monostearate, sucrose fatty acid ester, lecithin and mixtures thereof. In a specific embodiment, one or more of such emulsifiers may be present in an amount of about 0.01% to about 5.0%, by weight of the administered compositions. If the level of emulsifier is lower or higher than the said range, the emulsification cannot be realized, or wax value will rise.

Chewable compositions should begin to break and dissolve in the mouth shortly after chewing begins such that the compositions can be swallowed substantially as a solution. The dissolution profile of chewable compositions may be enhanced by including rapidly water-soluble fillers and excipients. Rapidly water-soluble fillers and excipients preferably dissolve within about 60 seconds of being wetted with saliva. Indeed, it is contemplated that if enough water-soluble excipients are included in the compositions of the present invention, they may become dissolvable rather than chewable composition forms. Examples of rapidly water soluble fillers suitable for use with the present invention include, by way of example and without limitation, saccharides, amino acids and the like. In a specific embodiment, the saccharide may be a mono-, di- or oligosaccharide. Examples of saccharides which may be added to the compositions of the present invention include, by way of example and without limitation, sorbitol, glucose, dextrose, fructose, maltose and xylitol (all monosaccharides); and sucrose, lactose, glucose, galactose and mannitol (all disaccharides). Other suitable saccharides are oligosaccharides. Examples of oligosaccharides are dextrates and maltodextrins. Other water soluble excipients that may be used with the present invention include, by way of example and without limitation, amino acids such as alanine, arginine, aspartic acid, asparagine, cysteine, glutamic acid, glutamine, glycine, histidine, isoleucine, leucine, lysine, methionine, phenylalanine, proline, serine, threonine, tryptophan, tyrosine and valine.

Disintegrants also may be included in the compositions of the present invention in order to facilitate dissolution. Disentegrants, including permeabilising and wicking agents, are capable of drawing water or saliva up into the compositions which promotes dissolution from the inside as well as the outside of the compositions. Such disintegrants, permeabilising and/or wicking agents that may be used in the present invention include, by way of example and without limitation, starches, such as corn starch, potato starch, pre-gelatinized and modified starches thereof, cellulosic agents, such as Ac-di-sol, montmorrilonite clays, cross-linked PVP, sweeteners, bentonite, microcrystalline cellulose, croscarmellose sodium, alginates, sodium starch glycolate, gums, such as agar, guar, locust bean, karaya, pectin, Arabic, xanthan and tragacanth, silica with a high affinity for aqueous solvents, such as colloidal silica, precipitated silica, maltodextrins, beta-cyclodextrins, polymers, such as carbopol, and cellulosic agents, such as hydroxymethylcellulose, hydroxypropylcellulose and hydroxyopropylmethylcellulose.

Finally, dissolution of the compositions may be facilitated by including relatively small particles sizes of the ingredients used.

In addition to those described above, any appropriate fillers and excipients may be utilized in preparing the swallowable, chewable and/or dissolvable compositions of the present invention so long as they are consistent with the objectives described herein. For example, binders are substances used to cause adhesion of powder particles in granulations. Such compounds appropriate for use in the present invention include, by way of example and without limitation, acacia, compressible sugar, gelatin, sucrose and its derivatives, maltodextrin, cellulosic polymers, such as ethylcellulose, hydroxypropylcellulose, hydroxypropylmethyl cellulose, carboxymethylcellulose sodium and methylcellulose, acrylic polymers, such as insoluble acrylate ammoniomethacrylate copolymer, polyacrylate or polymethacrylic copolymer, povidones, copovidones, polyvinylalcohols, alginic acid, sodium alginate, starch, pregelatinized starch, guar gum, polyethylene glycol and others known to those of ordinary skill in the art.

Diluents also may be included in the compositions of the present invention in order to enhance the granulation of the compositions. Diluents can include, by way of example and without limitation, microcrystalline cellulose, sucrose, dicalcium phosphate, starches, lactose and polyols of less than 13 carbon atoms, such as mannitol, xylitol, sorbitol, maltitol and pharmaceutically acceptable amino acids, such as glycin, and their mixtures.

Lubricants are substances used in composition formulations that reduce friction during composition compression. Lubricants that may be used in the present invention include, by way of example and without limitation, stearic acid, calcium stearate, magnesium stearate, zinc stearate, talc, mineral and vegetable oils, benzoic acid, poly(ethylene glycol), glyceryl behenate, stearyl fumarate, and others known to those of ordinary skill in the art.

Glidants improve the flow of powder blends during manufacturing and minimize composition weight variation. Glidants that may be used in the present invention include, by way of example and without limitation, silicon dioxide, colloidal or fumed silica, magnesium stearate, calcium stearate, stearic acid, cornstarch, talc and others known to those of ordinary skill in the art.

Colorants also may be included in the nutritional supplement compositions of the present invention. As used herein, the term “colorant” includes compounds used to impart color to pharmaceutical preparations. Such compounds include, by way of example and without limitation, FD&C Red No. 3, FD&C Red No. 20, FD&C Yellow No. 6, FD&C Blue No. 2, D&C Green No. 5, FD&C Orange No. 5, D&C Red No. 8, caramel, and ferric oxide, red and others known to those of ordinary skill in the art. Coloring agents also can include pigments, dyes, tints, titanium dioxide, natural coloring agents, such as grape skin extract, beet red powder, beta carotene, annato, carmine, turmeric, paprika and others known to those of ordinary skill in the art. It is recognized that no colorant is required in the nutritional supplement compositions described herein.

If desired, the compositions of the present invention may be sugar coated or enteric coated by standard techniques. The unit dose forms may be individually wrapped, packaged as multiple units on paper strips or in vials of any size, without limitation. The swallowable, chewable or dissolvable compositions of the present invention may be packaged in unit dose, rolls, bulk bottles, blister packs and combinations thereof, without limitation.

The swallowable, chewable or dissolvable compositions of the present invention may be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable nutritional supplements with improved mouthfeel. U.S. Pat. No. 5,965,162, relates to compositions and methods for preparing multi-vitamin comestible units which disintegrate quickly in the mouth, especially when chewed. Further, all pharmaceutical carriers and formulations described herein are well known to those of ordinary skill in the art, and determination of workable proportions in any particular instance will generally be within the capability of the person skilled in the art. Details concerning any of the excipients of the invention may be found in WADE & WALLER, HANDBOOK OF PHARMACEUTICAL EXCIPIENTS (2nd ed. 1994). All active ingredients, fillers and excipients are commercially available from companies such as Aldrich Chemical Co., FMC Corp, Bayer, BASF, Alexi Fres, Witco, Mallinckrodt, Rhodia, ISP, and others.

Other objectives, features and advantages of the present invention will become apparent from the following specific examples. The specific examples, while indicating specific embodiments of the invention, are provided by way of illustration only. Accordingly, the present invention also includes those various changes and modifications within the spirit and scope of the invention that may become apparent to those skilled in the art from this detailed description. The invention will be further illustrated by the following non-limiting examples.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLE 1

A composition of the following formulation was prepared in caplet form, including the appropriate excipients, by standard methods known to those of ordinary skill in the art:

| | | |
|---|---|---|
| Carotenoids | 3000 | IU |
| (Alpha-Carotene, Beta-Carotene, Cryptoxanthin, Lutein, | | |
| Zeaxanthin) | | |
| Vitamin E | 100 | IU |
| Vitamin D3 | 1000 | IU |
| Vitamin C (Ascorbic Acid) | 300 | mg |
| Vitamin B1 (Thiamine HCl) | 20 | mg |
| Vitamin B2 (Riboflavin) | 5 | mg |
| Niacin (Niacinamide) | 25 | mg |
| Folic Acid | 1 | mg |
| Vitamin B6 (Pyridoxine HCl) | 25 | mg |
| Biotin | 100 | μg |
| Pantothenic Acid (Calcium Pantothenate) | 15 | mg |
| Vitamin B12 (Cyanocobalamin) | 50 | μg |
| Magnesium (Magnesium Oxide) | 50 | mg |
| Manganese (Manganese Sulfate) | 1.5 | mg |
| Zinc (Zinc Oxide) | 25 | mg |
| Selenium (Sodium Selenate) | 100 | μg |
| Chromium (Chromium Chloride) | 50 | μg |
| Copper (Cupric Sulfate) | 1.5 | mg |
| Alpha Lipoic Acid | 15 | mg |
| Lutein | 5 | mg |

### EXAMPLE 2

A study is undertaken to evaluate the effectiveness of the composition of the present invention in the treatment of patients. The objective of the study is to determine whether oral intake of the composition results in an improvement of the nutritional status of the patient, either therapeutically or prophylacticly. A double-blind, placebo controlled study is conducted over a twelve-month period. A total of sixty subjects (30 men and 30 women), aged 40 to 85 years, suffering from dietary restrictions or a disease state such as anorexia, malnutrition, gastrointestinal disorders, chronic alcoholism, chronic infections, acute infections, congestive heart failure, hyperthyroidism, poorly controlled diabetes, cheilosis, gingivitis, sensitivity to iron, hemosiderosis, hemochromatosis, or stomatitis, or a propensity or disposition to such a disease state are chosen for the study. An initial assessment of nutritional status is conducted utilizing methods such as testing serum levels of 25-hydroxyvitamin D using a competitive binding assay to assess vitamin D deficiency, the peroxide hemolysis test to assess vitamin E deficiency, measurement of erythrocyte transketolase activity to determine thiamine levels, determination of erytirocyte glutathione reductase activity to assess riboflavin status, and high performance liquid chromatography to directly measure PLP and pyridoxine levels.

The sixty subjects are separated into two separate groups of fifteen men and fifteen women. In the first group, each subject is administered 1 to 2 caplets, daily, of the composition as described in Example 1. In the second group (control), each subject is administered 1 to 2 placebo caplets, daily

An assessment of nutritional status for each subject is measured at one-month intervals for a twelve month period as described above. Specifically, in regard to vitamin D deficiency, the serum levels of 25-hydroxyvitamin D are measured to assess if they meet levels as high as 30 ng/ml or are increasing towards a level of 30 ng/ml. The data is evaluated using multiple linear regression analysis and a standard students t-test. In each analysis the baseline value of the outcome variable is included in the model as a covariant. Treatment by covariant interaction effects is tested by the method outlined by Weigel & Narvaez, 12 CONTROLLED CLINICAL TRIALS 378-94 (1991). If there are no significant interaction effects, the interaction terms are removed from the model. The regression model assumptions of normality and homogeneity of variance of residuals are evaluated by inspection of the plots of residuals versus predicted values. Detection of the temporal onset of effects is done sequentially by testing for the presence of significant treatment effects at 16, 12, and 8 weeks, proceeding to the earlier time in sequence only when significant effects have been identified at each later time period. Changes from the baseline within each group are evaluated using paired t-tests. In addition, analysis of variance is performed on all baseline measurements and measurable subject characteristics to assess homogeneity between groups. All statistical procedures are conducted using the Statistical Analysis System (SAS Institute Inc., Cary, N.C.). An alpha level of 0.05 is used in all statistical tests.

A statistically significant improvement in the nutritional status is preferably observed in the treated subjects upon completion of the study over the controls. Specifically, in regard to vitamin D deficiency, the tested serum levels of 25-hydroxyvitamin D are preferably in the range of 30 ng/ml for treated subjects. The study may also look at the progression of the disease state, or the prevention or delay of a disease or disease state, or the reduction of the severity of a disease. The differences between nutritional state or the progression of the disease state, or the prevention or delay of a disease or disease state, or the reduction of the severity of a disease, between the treated subjects and controls are preferably statistically significant and or observable by clinical or other tests or evaluations. Therefore, the study confirms that oral administration of the composition of the present invention is effective as a nutritional supplement, either therapeutically or prophylacticly, for example, in preventing the severity or delaying or preventing the onset of a disease.

While there has been described what is presently believed to be the preferred embodiments of the present invention, other and further modifications and changes may be made without departing from the spirit of the invention. All farther and other modifications and changes are included that come within the scope of the invention as set forth in the claims. The disclosure of all publications cited above are expressly incorporated by reference in their entireties to the same extent as if each were incorporated by reference individually.

What is claimed is:

1. A composition comprising carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, and lutein.

2. The composition of claim 1, wherein said composition further comprises one or more pharmaceutically acceptable carriers.

3. The composition of claim 1, wherein said carotenoids comprise one or more carotenoids selected from the group consisting of alpha-carotene, beta-carotene, cryptoxanthin, lutein, and zeaxanthin.

4. The composition of claim 3, wherein said carotenoids include lutein and zeaxanthin.

5. The composition of claim 1, wherein said vitamin D comprises vitamin D3.

6. The composition of claim 1, wherein said vitamin B12 comprises cobalamin.

7. The composition of claim 1, wherein said vitamin B6 comprises pyridoxine.

8. The composition of claim 1, wherein said vitamin B1 comprises thiamin.

9. The composition of claim 1, wherein said vitamin B2 comprises riboflavin.

10. The composition of claim 1, wherein said vitamin B3 comprises niacin.

11. The composition of claim 1, wherein said vitamin B5 comprises pantothenic acid.

12. The composition of claim 1, wherein said vitamin D is present in the range of about 500 IU to about 1500 IU.

13. The composition of claim 1, wherein said vitamin D is present in the amount of about 1000 IU.

14. A composition comprising carotenoids, vitamin E, more than 480 IU vitamin D3, vitamin C, folio acid, pyridoxine, cobalamin, thiamin, riboflavin, niacin, pantothenic acid, biotin, magnesium, manganese, zinc, selenium, chromium, copper, alpha lipoic acid, and lutein.

15. The composition of claim 14, wherein said composition comprises about 1500 IU to about 4500 IU carotenoids, about 50 IU to about 150 IU vitamin E, about 500 IU to about 1500 IU vitamin D3, about 150 mg to about 450 mg vitamin C, 0.5 mg to about 1.5 mg folic acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein.

16. The composition of claim 14, wherein said composition comprises about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, and about 5 mg lutein.

17. The composition of claim 16, wherein said composition further comprises one or more pharmaceutical carriers and is administrable to a patient.

18. The composition of claim 17, wherein said composition is administrable to said patient orally.

19. A method comprising administering to a patient the composition of claim 1.

20. The method of claim 19, wherein said composition further comprises one or more pharmaceutically acceptable carriers.

21. The method of claim 19, wherein said carotenoids comprise one or more carotenoids selected from the group consisting of alpha-carotene, beta-carotene, cryptoxanthin, lutein, and zeaxanthin.

22. The method of claim 21, wherein said carotenoids include lutein and zeaxanthin.

23. The method of claim 19, wherein said vitamin D comprises vitamin D3.

24. The method of claim 19, wherein said vitamin B12 comprises cobalamin.

25. The method of claim 19, wherein said vitamin B6 comprises pyridoxine.

26. The method of claim 19, wherein said vitamin B1 comprises thiamin.

27. The method of claim 19, wherein said vitamin B2 comprises riboflavin.

28. The method of claim 19, wherein said vitamin B3 comprises niacin.

29. The method of claim 19, wherein said vitamin B5 comprises pantothenic acid.

30. The method of claim 19, wherein said vitamin D is present in the range of about 500 IU to about 1500 IU.

31. The method of claim 19, wherein said vitamin D is present in the amount of about 1000 IU.

32. A method comprising administering to a patient the composition of claim 14.

33. The method of claim 32, wherein said composition comprises about 1500 IU to about 4500 IU carotenoids, about 50 IU to about 150 IU vitamin E, about 500 IU to about 1500 IU vitamin D3, about 150 mg to about 450 mg vitamin C, 0.5 mg to about 1.5 mg folic acid, about 12.5 mg to about 37.5 mg pyridoxine, about 25 μg to about 75 μg cobalamin, about 10 mg to about 30 mg thiamin, about 2.5 mg to about 7.5 mg riboflavin, about 12.5 mg to about 37.5 mg niacin, about 7.5 mg to about 22.5 mg pantothenic acid, about 50 μg to about 150 μg biotin, about 25 mg to about 75 mg magnesium, about 0.75 mg to about 2.25 mg manganese, about 12.5 mg to about 37.5 mg zinc, about 50 μg to about 150 μg selenium, about 25 μg to about 75 μg chromium, about 0.75 mg to about 2.25 mg copper, about 7.5 mg to about 22.5 mg alpha lipoic acid, and about 2.5 mg to about 7.5 mg lutein.

34. The method of claim 32, wherein said composition comprises about 3000 IU carotenoids, about 100 IU vitamin E, about 1000 IU vitamin D3, about 300 mg vitamin C, about 1.0 mg folic acid, about 25 mg pyridoxine, about 50 μg cobalamin, about 20 mg thiamin, about 5 mg riboflavin, about 25 mg niacin, 15 mg pantothenic acid, 100 μg biotin, about 50 mg magnesium, about 1.5 mg manganese, about 25 mg zinc, about 100 μg selenium, about 50 μg chromium, about 1.5 mg copper, about 15 mg alpha lipoic acid, and about 5 mg lutein.

35. The method of claim 34, wherein said composition further comprises one or more pharmaceutical carriers and is administered to a patient.

36. The method of claim 35, wherein said composition is administered to said patient orally.

37. A composition consisting of carotenoids, vitamin E, more than 480 IU vitamin D, vitamin C, folic acid, vitamin B6, vitamin B12, vitamin B1, vitamin B2, vitamin B3, vitamin B5, biotin, magnesium, manganese, zinc, selenium, chromium, copper, alpha lipoic acid, lutein and one or more pharmaceutically acceptable carriers.

38. A method consisting of administering to a patient the composition of claim 37.
